(19) United States
(12) Patent Application Publication
(10) Pub. No.: US 2024/0009093 A1
(43) Pub. Date: Jan. 11, 2024

(54) COMPOSITIONS COMPRISING AN IODIDE OR THIOCYANATE, AND METHODS FOR TREATING TEETH

(71) Applicant: 3M INNOVATIVE PROPERTIES COMPANY, St. Paul, MN (US)

(72) Inventors: Yizhong WANG, Woodbury, MN (US); Ahmed S. ABUELYAMAN, Woodbury, MN (US)

(21) Appl. No.: 18/255,411

(22) PCT Filed: Dec. 2, 2021
(86) PCT No.: PCT/IB2021/061257

§ 371 Date: Jun. 3, 2023

Related U.S. Application Data
(60) Provisional application No. 63/199,029, filed on Dec. 3, 2020.

(51) Int. Cl. A61K 8/20; A61K 8/22; A61K 8/46; A61K 8/41; A61Q 11/00

(52) U.S. Cl. CPC A61K 8/20; A61K 8/22; A61K 8/46; A61K 8/41; A61Q 11/00; A61K 2800/884

(57) ABSTRACT

A method of treating a tooth of a patient includes introducing an oxidizing composition into an oral cavity of the patient such the oxidizing composition contacts the tooth. The method further includes introducing an oral care composition into the oral cavity such that the oral care composition contacts the tooth, wherein the oral care composition comprises an iodide or thiocyanate.

### FIELD

The present invention generally relates to compositions and methods for removing or mitigating the appearance of stains on teeth. The compositions and methods may be used, for example, to remove stains that result from the use of silver containing compounds to treat teeth.

### DETAILED DESCRIPTION

Silver diamine fluoride (SDF) has proven an effective treatment for addressing tooth hypersensitivity and arresting dental caries. Use of SDF, however, is associated with dark staining of the teeth.

There are two mechanisms by which SDF (and other silver containing compounds) result in dark or black staining of teeth. The first, light induced silver oxide, results from the compound reacting with the tooth to form silver phosphate precipitations which, when exposed to light, convert to silver oxide (which is black in color). The second, microbial induced silver sulfide, results from sulfide compounds produced by some of the microbial species in the oral cavity reacting with the silver phosphate precipitations to form silver sulfide (which turns black in color over time).

Consequently, compositions and methods for reversing, eliminating, or mitigating the dark stains caused from oral use of SDF (or other silver containing compounds) may be desirable.

In this application, terms such as “a,” “an,” and “the” are not intended to refer to only a singular entity, but include the general class of which a specific example may be used for illustration. The terms “a,” “an,” and “the” are used interchangeably with the phrases “at least one” and “one or more.” The phrases “at least one of” and “comprises at least one of” followed by a list refers to any one of the items in the list and any combination of two or more items in the list.

The term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about”. As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Generally, the present disclosure relates to methods for removing or mitigating the presence of tooth staining that results from use of silver containing compounds in the oral cavity. Surprisingly, it was discovered that by performing an oxidizing treatment following by introduction of a source of iodide or thiocyanate into the oral cavity, such stains can be removed or substantially mitigated.

In some embodiments, the methods of the present disclosure may include introducing into the oral cavity an oxidizing composition (such that the composition contacts one or more teeth to be treated). Generally, the oxidizing composition (or solution) may be any composition that is capable of oxidizing silver sulfides that are present on a tooth surface. In some embodiments, the oxidizing solution may include any organic or inorganic compound having one or more peroxide groups. Such compounds may include hydrogen peroxide, calcium peroxide, sodium peroxide, benzol peroxide, peracetic acid and related salts, perester, potassium persulfate, ammonium persulfate, calcium persulfate, sodium persulfate, or combinations thereof. In some embodiments, peroxide group containing compounds (or peroxides) may be present in the oxidizing composition in an amount of at least 0.1 wt. %, at least 0.5 wt. %, or at least 1.0 wt. %, based on the total weight of the oxidizing composition. In some embodiments, the oxidizing composition may be an aqueous solution that includes at least 30 wt. %, at least 40 wt. %, or at least 50 wt. % water, based on the total weight of the oxidizing composition.

In some embodiments, the step of introducing the oxidizing composition may include contacting one or more teeth to be treated with the oxidizing composition for at least 5 seconds, at least 10 seconds, at least 30 seconds, at least 1 minute, at least 5 minutes, at least 15 minutes, at least 30 minutes, at least 1 hour, at least 2 hours, or at least 5 hours.

In some embodiments, following the oxidizing step, the methods may include rinsing the oral cavity with a fluid (e.g., water) to facilitate removal of any undesirable substances or debris from the tooth surface or oral cavity.

In some embodiments, following the step of oxidizing (or concomitantly or nearly concomitantly with the step of oxidizing), (and optionally rinsing with water) the methods of the present disclosure may include introducing into the oral cavity an oral care composition (or solution) that includes an iodide (or iodide ions) or a thiocyanate (or thiocyanate ions). For example, iodide may be present in the oral care composition as potassium iodide, ammonium iodide, sodium iodide, silver iodide, calcium iodide, combinations thereof. Thiocyanate may be present as ammonium thiocyanate, sodium thiocyanate, potassium thiocyanate, guanidinium thiocyanate, or combinations thereof.

In some embodiments, iodide or thiocyanate may be present in the oral care composition in an amount of at least 5 wt. %, at least 10 wt./q, at least 30 wt./, or at least 50 wt./, based on the total weight of the oral care composition. In some embodiments, the oral care composition may be saturated with iodide or thiocyanate (i.e., iodide or thiocyanate may be present in an amount that represents a maximum amount that iodide or thiocyanate can be present in a particular solution).

In some embodiments, the step of introducing a source of iodide may include introducing a composition that, in addition to providing a source of iodide, may contribute to addressing tooth hypersensitivity and arresting dental caries. In such embodiments, the iodide containing oral care composition may be an iodide containing aqueous composition (e.g., solution) that includes silver cations, iodide anions, fluoride anions, and water.

In some embodiments, the source of silver cations in the iodide containing aqueous composition may be selected from silver fluoride, silver chloride, silver nitrate, silver iodide, silver diamine fluoride, and combinations thereof.

In some embodiments, the silver ions (also referred to herein as silver cations) may be present in the iodide containing aqueous composition in an amount of at least 12.2 percent by weight (wt-%), at least 13 wt-%, or at least 13.5 wt-%, wherein the weight percentages are based on the total weight of the composition (e.g., solution). In some embodiments, the silver cations may be present in the composition in an amount of up to 20 wt-%, up to 19 wt-%, up to 18 wt-%, or up to 17 wt-%, wherein the weight percentages are based on the total weight of the composition (e.g., solution).

In some embodiments, the source of fluoride anions in the iodide containing aqueous composition may be selected from silver fluoride, silver diamine fluoride, sodium fluoride, ammonium fluoride, potassium fluoride, amine fluoride, and combinations thereof.

In some embodiments, the fluoride ions (also referred to herein as fluoride anions) in the iodide containing aqueous compositions may be present in an amount of at least 2.0 wt-%, at least 2.1 wt-%, at least 2.2 wt-%, or at least 2.25 wt-%, wherein the weight percentages are based on the total weight of the composition (e.g., solution). In some embodiments, the fluoride anions may be present in an amount of up to 4.0 wt-%, up to 3.9 wt-%, up to 3.8 wt-%, up to 3.5 wt-%, or up to 3.0 wt-%, wherein the weight percentages are based on the total weight of the composition (e.g., solution).

In some embodiments, the source of iodide ions (also referred to herein as iodide anions) may be selected from ammonium iodide, sodium iodide, potassium iodide, silver iodide, and combinations thereof. In some embodiments, the source of iodide ions may include ammonium iodide and optionally a secondary source of iodide ions selected from sodium iodide, potassium iodide, silver iodide, and combinations thereof. In some embodiments, the source of iodide ions may include ammonium iodide and a secondary source of iodide ions selected from sodium iodide, potassium iodide, silver iodide, and combinations thereof.

In some embodiments, the molar ratio of silver to iodide ions in the composition may be less than 0.42:1. In some embodiments, the molar ratio of silver to iodide ions in the composition may be at least 0.09:1.

In some embodiments, the iodide containing aqueous composition (e.g., solution) may include: 12.25-20 wt-% silver cations and 2.0-4.0 wt-% fluoride anions; or 13-17 wt-% silver cations; and 2.25-3.0 wt-% fluoride anions, wherein the weight percentages are based on the total weight of the composition (e.g., solution).

In some embodiments, the iodide containing aqueous composition (e.g., solution) may include water in an amount of at least 20 wt-%, based on the total weight of the composition (e.g., solution). In some embodiments, the amount of water in the aqueous composition (e.g., solution) may be less than 41.5 wt-%, based on the total weight of the composition (e.g., solution).

In some embodiments, an aqueous oral care solution includes: silver cations; thiocyanate anions; fluoride anions; and water.

In some embodiments, the silver ions (also referred to herein as silver cations) may be present in the thiocyanate containing aqueous composition in an amount of at least 12.25 percent by weight (wt-%), at least 13 wt-%, or at least 13.5 wt-%, wherein the weight percentages are based on the total weight of the composition (e.g., solution). In some embodiments, the silver cations may be present in the thiocyanate containing composition in an amount of up to 20 wt-%, up to 19 wt-%, up to 18 wt-%, or up to 17 wt-%, wherein the weight percentages are based on the total weight of the composition (e.g., solution).

In some embodiments, the source of silver cations in the thiocyanate containing aqueous composition may be selected from silver fluoride, silver chloride, silver nitrate, silver iodide, silver diamine fluoride, and combinations thereof.

In some embodiments, the source of fluoride anions in the thiocyanate containing aqueous composition may be selected from silver fluoride, silver diamine fluoride, sodium fluoride, ammonium fluoride, potassium fluoride, amine fluoride, and combinations thereof.

In some embodiments, the fluoride ions (also referred to herein as fluoride anions) in the thiocyanate containing aqueous compositions may be present in an amount of at least 2.0 wt-%, at least 2.1 wt-%, at least 2.2 wt-%, or at least 2.25 wt-%, wherein the weight percentages are based on the total weight of the composition (e.g., solution). In some embodiments, the fluoride anions may be present in an amount of up to 4.0 wt-%, up to 3.9 wt-%, up to 3.8 wt-%, up to 3.5 wt-%, or up to 3.0 wt-%, wherein the weight percentages are based on the total weight of the composition (e.g., solution).

In some embodiments, a thiocyanate containing aqueous oral care solution includes: 12.2-20 wt-% silver cations; and 2.0-4.0 wt-% fluoride anions; wherein the weight percentages are based on the total weight of the solution. In some embodiments, the thiocyanate containing aqueous oral care solution includes: 12.2-20 wt-% silver cations; and 2.2-3.5 wt-% fluoride anions.

In some embodiments, the molar ratio of silver to thiocyanate ions is less than 0.37:1. In certain embodiments, the molar ratio of silver to thiocyanate ions is at least 0.1:1. In certain embodiments, the source of thiocyanate ions (also referred to herein as thiocyanate anions) is selected from ammonium thiocyanate, sodium thiocyanate, potassium thiocyanate, guanidinium thiocyanate, and combinations thereof.

In certain embodiments, a thiocyanate containing aqueous oral care solution includes water in an amount of at least 20 wt-% and less than 57.5 wt-%, based on the total weight of the solution.

Upon contact with additional water or saliva in the oral environment, the oral care solution may form a precipitate (i.e., a solid formed from the solution). Regarding the thiocyanate containing aqueous oral care solution, the precipitate includes AgSCN. The resultant silver compound provides antibacterial effect. While not being bound by theory, it is believed that the AgSCN, fluoride ions, and excess thiocyanate ions, complex with calcium in the tooth.

In some embodiments, a thiocyanate containing aqueous oral care solution includes: 12.2-20 wt-% silver cations; and 2.0-4.0 wt-% (or 2.2-3.5 wt-%) fluoride anions; wherein the weight percentages are based on the total weight of the solution; and thiocyanate anions, wherein a molar ratio of silver to thiocyanate ions is at least 0.1:1 and less than 0.37:1; wherein the oral care solution forms a precipitate upon contact with additional water or saliva.

In some embodiments, the step of introducing the source of iodide or thiocyanate may include contacting one or more teeth to be treated with the composition for at least 10 seconds, at least 30 seconds, at least 1 minute, at least 2 minutes, or at least 5 minutes. In some embodiments, the step of introducing the source of iodide or thiocyanate may occur within 1 second, within 5 seconds, within 10 seconds, within 20 seconds, within 30 seconds, within 1 minute, within 10 minutes, within 30 minutes, or within 60 minutes of the step of introducing the oxidizing composition.

In some embodiments, following the step of introducing a source of an iodide or thiocyanate, the methods of the present disclosure may optionally include rinsing the oral cavity with a fluid (e.g., water) to facilitate removal of any undesirable substances or debris from the tooth surface or oral cavity.

As discussed above, the methods of the present disclosure may be useful for addressing both light induced silver oxide and microbial induced silver sulfide. In some embodiments, the mechanism of treatment for each of these may be understood to be as follows:

### Light Induced Silver Oxide

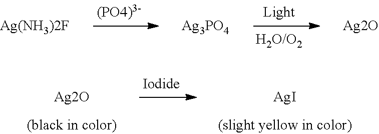

### Microbial Induced Silver Sulfide

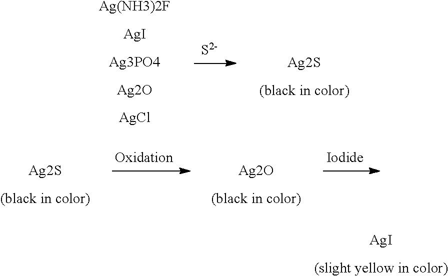

Generally, it is believed that the silver iodide products that result from some methods of the present disclosure (which may be in the form of a precipitate) along with any fluoride ions and any excess iodide ions may complex with calcium in the tooth, and thereby avoid discoloration (even when exposed to light). Significantly, such products do not turn (discolor) to a dark color such as black, brown, or grey after being precipitated and exposed to light.

In some embodiments, as discussed above, certain iodide or thiocyanate containing compositions of the present disclosure may be aqueous compositions (e.g., solutions). Such aqueous compositions may also include an amount (e.g., a minor amount) of one or more organic solvents. Examples of suitable organic solvents include ethanol, isopropanol, dimethyl sulfoxide (DMSO), isoprene sulfone (IS), butadiene sulfone (BS), piperylene sulfone (PS), ethyl acetate, methyl acetate, isopropyl acetate, acetone, methyl ethyl ketone (MEK), methyl isobutyl ketone (MIBK), and combinations thereof.

In some embodiments, any of the above described iodide or thiocyanate containing compositions may include one or more additional active agents. When included, the one or more additional active agents may include one or more active agents that are active in the oral cavity against disorders, diseases, or conditions of the teeth, gums, cheeks, tongue, roof of the mouth, and the like.

Examples of additional active agents that can be employed include one or more fluorine-containing compounds, such as sodium monofluorophosphate, stannous fluoride, calcium fluoride, strontium fluoride, zinc fluoride, zinc potassium fluoride, ammonium fluoride, potassium magnesium fluoride, and combinations thereof.

Examples of additional active agents that can be employed include one or more whitening agents, anticalculus agents, remineralization agents, stannous sources, antimicrobial agents, antioxidants, saliva stimulating agents, breath freshening agents, antiplaque agents, anti-inflammatory agents, H₂ antagonists, desensitizing agents, nutrients, and proteins. Various combinations of such additional active agents may be used, if desired. When employed, one or more additional active agents will be typically used in amounts sufficient to achieve their intended effect.

When employed, the whitening agents can be a wide variety of suitable whitening agents. The whitening agents can include, for example, a peroxide whitening agent, a non-peroxide whitening agent, or both. Peroxide whitening agents include hydrogen peroxide, peroxide of alkali or alkaline earth metals, such as sodium peroxide, potassium peroxide, lithium peroxide, magnesium peroxide, calcium peroxide, barium peroxide, and the like, glyceryl hydrogen peroxide, alkyl hydrogen peroxide, dialkyl peroxide, peroxy acids or peroxy acid salts, benxoyl peroxide, urea peroxide, and the like. Hydrogen peroxide is most common. Non-peroxide whitening agents include chlorine dioxide, chlorites, and hypochlorites. Chlorites and hyperchlorites are typically in the form of alkali or alkaline earth metal salts, such as salts of lithium, potassium, sodium, magnesium, calcium, or barium. Colorants, titanium dioxide, and hydroxyapatite can also be used.

When employed, the anticalculus agents can be a wide variety of suitable anticalculus agents. The anticalculus agents can include, for example, phosphates, polyphosphates, such as pyrophosphates, polyolefin sulfonates, polyolefin phosphates, diphosphonates, phosphonoalkane carboxylic acids, and salts thereof, typically alkali metal or ammonium salts.

When employed, the remineralization agents can be a wide variety of suitable remineralization agents. The remineralization agents can include, for example, materials that release calcium ions, phosphorous-containing ions, or both, such as calcium phosphate (e.g., mono-, di-, and/or tricalcium phosphate), hydroxyapatite, calcium carbonate, and the like.

Examples of materials that release calcium ions are calcium salts that are water soluble, such as those selected from calcium chloride, calcium nitrate, calcium gluconate, calcium lactate gluconate, calcium acetate, hydrates thereof, and combinations thereof. In certain embodiments, the calcium salt is selected from calcium chloride, calcium nitrate, hydrates thereof, and combinations thereof.

When employed, the stannous sources can be a wide variety of suitable sources of stannous ions. The stannous ion sources can include, for example, stannous halides, organic stannous carboxylate salts, such as stannous formate, stannous acetate, stannous gluconate, stannous lactate, stannous tartrate, and stannous citrate. When the fluoride source is stannous fluoride, it can also function as a stannous source.

When employed, the antimicrobial agents can include a wide variety of orally acceptable antimicrobial agents. Examples include triclosan, 8-hydroxyquinoline, zinc ion, stannous ion, cupric compounds, phthalic acid and salts thereof, quaternary ammonium compounds, sanguinarine, salicylanilide, salicylic acid, thymol, eugenol, neomycin, kanamycin, clindamycin, amoxicillin, tetracycline, doxycycline, minocycline, metronidazole, chlorohexidine, and the like.

When employed, the antioxidants can be a wide variety of orally acceptable antioxidants. Examples include butylated hydroxy anisone, butylated hydroxy toluene, vitamin A, carotenoids, vitamin E, flavonoids, polyphenols, ascorbic acid or salts thereof, chlorophyll, melatonin, and the like.

When employed, the saliva stimulants can be a wide variety of orally acceptable saliva stimulants. Examples include citric acid, lactic acid, succinic acid, ascorbic acid, adipic acid, fumaric acid, and tartaric acid.

When employed, the breath freshening agents can be a wide variety of orally acceptable breath freshening agents. Examples include zinc salts such as zinc salts of gluconate, citrate, chlorite, alpha-ionone, and the like.

When employed, the antiplaque agents can be a wide variety of orally acceptable antiplaque agents. Examples include stannous salts, salts of copper, magnesium or strontium, dimethicone copolyols, such as cetyl dimethicone copolyol, papain, glucamylase, glucose oxidase, urea, calcium lactate, calcium glycerophosphate, strontium polyacrylates, and the like. Further examples of antiplaque agents include biofilm inhibition agents, particularly those described in U.S. Pat. No. 8,968,709 (Yang et al.).

When employed, the anti-inflammatory agents can be a wide variety of orally acceptable anti-inflammatory agents. Examples include steroids such as flucinolone and hydrocortisone, non-steroidal anti-inflammatory drugs such as ketorolac, flurbiprofen, ibuprofen, naproxen, indomethacin, diclofenac, etodolac, indomethacin, sulindac, tomlmetin, ketoprofen, fenoprofen, piroxicam, nabumetone, acetyl salicylic acid, salicylic acid, diflunisal, meclofenamate, mefenamic acid, oxyphenbutazone, phenylbutazone, and the like.

When employed, the H₂ antagonists can be a wide variety of orally acceptable H₂ antagonists. Examples include cimetidine, etinidine, ranitidine, tiotidine, lupitidine, denetidine, famotidine, roxatidine, pifatidine, lamtidine, zaltidine, nizatidine, mifentidine, ramixotidine, loxtidine, bisfentidine, sufotidine, ebrotidine, impromdine, and the like.

When employed, the desensitizing agents can be a wide variety of orally acceptable desensitizing agents. Examples include potassium citrate, potassium chloride, potassium tartrate, potassium bicarbonate, potassium oxalate, potassium nitrate, strontium salts, arginine, acetyl salicylic acid or salts thereof, salicylic acid or salts thereof, codeine, acetaminophen, and the like.

When employed, the nutrients can be a wide variety of orally acceptable nutrients. Examples include vitamins, such as vitamins C, D, thiamine, riboflavin, folic acid, nicotinamide, niacin, pyridoxine, bioflavonoids, and the like, supplements, such as amino acids, lipotropics, fish oil, polyunsaturated fatty acids, eicosapentanoic acid, docosahexanic acid, coenzyme Q10, ubiquinone, minerals such as potassium, and the like.

When employed, the proteins can include a wide variety of orally acceptable proteins. Examples include milk proteins, peroxide producing enzymes, amylase, papain, glucoamylase, glucose oxidase, and the like.

In some embodiments, any of the above described iodide or thiocyanate containing compositions may include a pharmaceutically acceptable buffer. The type and amount of such buffer may be selected to provide an oral care composition (e.g., solution) with a pH of at least 5.5, at least 6, or at least 6.5. In certain embodiments, the type and amount of such buffer is selected to provide an oral care composition (e.g., solution) with a pH of up to 9, up to 8.5, up to 7.5, or up to 7. In certain embodiments, the type and amount of such buffer is selected to provide an oral care composition (e.g., solution) with a pH of 6.5 to 7.5, or a pH of 7.0. A wide variety of suitable pharmaceutically acceptable buffers can be included. Examples include acetate (e.g., sodium acetate), sodium carbonate, citrate (e.g., sodium citrate), tartrate, glycylglycine, histidine, glycine, lysine, arginine, sodium dihydrogen phosphate, disodium hydrogen phosphate, sodium phosphate, tris(hydroxymethyl)-aminomethane, or mixtures thereof.

In some embodiments, any of the above described iodide or thiocyanate containing compositions may include a thickener to provide a composition (e.g., solution) with a suitable viscosity to allow for the desired method of application. For example, a suitable thickener in a sufficient amount may be used to achieve a composition (e.g., solution) viscosity adequate to maintain the composition (e.g., solution) in an inverted mouthpiece tray applicator for up to four minutes (typical time for a professionally applied fluoride treatment), and yet be fluid enough to have acceptable handling characteristics for the dental operator (e.g., when dispensing into a dental tray applicator). Or, a suitable thickener in a sufficient amount may be used to achieve a viscosity adequate to paint on a tooth surface.

In certain embodiments, the type and amount of thickener is selected to provide an oral care composition (e.g., solution) with a viscosity of at least 0.5 Pascal seconds at a shear rate of 1.0/second. In certain embodiments, a type and amount of thickener is selected to provide an oral care composition (e.g., solution) with a viscosity of up to 500 Pascal seconds at a shear rate of 1.0/second.

In certain embodiments, a thickener is present in an oral care composition (e.g., solution) in an amount of less than 2.5 wt-%, based on the total weight of the aqueous composition (e.g., solution). In certain embodiments, a thickener is present in an amount of at least 0.5 wt-%, based on the total weight of the aqueous composition (e.g., solution).

Suitable thickeners are typically those that are generally safe for human ingestion (FDA approved for internal use), do not bind fluoride ions, and do not significantly affect the bioavailability of fluoride ions.

In certain embodiments, the thickener may be selected from natural gums, non-acid cellulose derivatives (e.g., hydroxyethyl cellulose), inorganic fillers (e.g., colloidal silica, fumed silica, alumina, titania, and zinc oxide), alkylene oxide polymers (e.g., polyethylene glycol, polypropylene glycol, and copolymers of polyethylene glycol and polypropylene glycol), non-acid modified starches, and combinations thereof.

In certain embodiments, aqueous oral care compositions (e.g., solutions) of the present disclosure may include one or more optional additives including flavoring agents (i.e., flavorants) and sweeteners. Other optional additives include surfactants. Various combinations of such additives may be used if desired.

In certain embodiments, aqueous oral care compositions (e.g., solutions) of the present disclosure may include a sweetener. A wide variety of orally acceptable sweeteners can be used. Common sweeteners include xylitol, sorbitol, sucralose, aspartame, saccharin, usually sodium saccharine, and the like. When present, a sweetener can be used in any suitable amount, most often in an amount sufficient to impart a pleasant sweetness to the composition (e.g., solution). The suitable amount is typically 0.5 wt-% to 15 wt-%, based on the total weight of the aqueous composition (e.g., solution).

In certain embodiments, aqueous oral care compositions (e.g., solutions) of the present disclosure may include a flavoring agent. A wide variety of orally acceptable flavoring agents can be used. Common flavoring agents include peppermint oil, spearmint oil, cherry flavor, citric acid, orange flavor, vanilla, strawberry flavor, coconut flavor, and bubble gum flavor. When present, a flavoring agent can be used in any suitable amount, most often in an amount sufficient to impart a desired flavor to the composition (e.g., solution). The suitable amount is typically 1 wt-% to 4 wt-%, based on the total weight of the aqueous composition (e.g., solution).

In certain embodiments, aqueous oral care compositions (e.g., solutions) of the present disclosure may include a surfactant. Typically, such surfactant is an anionic surfactant, examples of which include polysorbate, glycerol, polyglycerol-based surfactant, or combinations thereof. When present, a surfactant can be used in any suitable amount, most often in an amount sufficient to impart wettability. A suitable amount is typically 0.1 wt-% to 5.0 wt-%, based on the total weight of the aqueous composition (e.g., solution).

In some embodiments, any of the above-described compositions (e.g., oxidizing compositions, oral care compositions, aqueous oral care compositions) may be introduced into the oral cavity and applied to the teeth in any conventional fashion. For example, in some embodiments, any or all of the compositions may be applied by providing the patient with an amount of the composition and directing the patient to rinse or gargle with the composition for a recommended period. In some embodiments, any or all of the compositions may be applied by spraying the composition on a patient's teeth. In some embodiments, any or all of the compositions may be applied by painting or brushing the compositions on a patient's teeth. In some embodiments, any or all of the compositions may be applied by dispensing the compositions into a dental or orthodontic tray and bringing the dental or orthodontic tray into contact with a patient's teeth. In some embodiments, any or all of the compositions may be incorporated into a dental fluoride varnish and applied by conventional varnish methods. Most fluoride varnishes on the market are rosin/ethanol based formulations with a hydrophobic nature. The varnish is painted on the teeth and remains in place for several hours to allow for the fluoride to be released from the composition. Typically, dentists use fluoride varnishes for in-office fluoride treatment. Most dental fluoride varnishes include 5% sodium fluoride. The dose of varnish may be about 0.5 grams.

In some embodiments, any or all of the compositions of the present disclosure may be in the form of delivered via a liquid, an emulsion, a paste, a gel (or gel coating), a tooth whitening strip, tooth paste, chewing gum, or combinations thereof.

In some embodiments of the methods described above, the method may further include placing a dental restorative on the tooth surface having the oral care composition (e.g., solution) applied thereto (either before or after the composition (e.g., solution) is dried, rinsed off, wiped off, and/or a precipitate is formed on the tooth surface). Examples of dental restorative include, but are not limited to, an adhesive (such as 3M SCOTCHBOND Universal Adhesive (available from 3M Company of St. Paul, MN, USA), primer, cement (such as 3M RelyX UNICEM 2 AUTOMIX Self-Adhesive Resin Cement, available from 3M Company of St. Paul, MN, USA), liner (such as 3M ESPE VITREBOND Plus Light Cure Glass Ionomer Liner/Base), sealant, amalgam, resin, resin composite (3M FILTEK Z250 Universal Restorative), glass ionomer (such as 3M KETAC Universal APLICAP Glass Ionomer Restorative), resin-modified glass ionomer (such as RelyX Luting Plus RMGI Cement), glass-ceramic, ceramic, metal, plastic, or combination thereof.

As discussed above, the methods of the present disclosure are intended to remove or mitigate tooth stains or dark discolorations that result from the use of silver containing compounds to treat teeth. In this regard, in some embodiments, prior to employing the methods of the present disclosure, the patient's teeth may have already undergone a treatment that included exposing the teeth to a silver containing compound (e.g., silver diamine fluoride). Consequently, in some embodiments, prior to the steps of introducing an oxidizing composition and introducing an oral care composition, the patient's teeth may include one or more stains or dark spots caused by exposure to a silver containing compound.

In some embodiments, the present disclosure is directed to a kit that includes either or both of any of the above-described oxidizing compositions and oral care compositions and an applicator. The applicator may include a dental brush, cotton tip swab, or the like. In some embodiments, the applicator may be integrated into a container having any of the above-described compositions therein. The kits may further include one or more of a dental restorative, a tray, a dish, a well, or a pan. Examples of dental restoratives include an adhesive, primer, cement, liner, sealant, amalgam, resin, resin composite, glass ionomer, resin-modified glass ionomer, glass-ceramic, ceramic, metal, plastic, or combination thereof.

The compositions (e.g., oral care compositions) of the present disclosure can be made using any techniques known to one of skill in the art. In some embodiments, the components are added together into water and dissolved, in no particular order. Alternatively, the order of addition can be important in obtaining a composition (e.g., solution). For example, in embodiments in which the aqueous oral care compositions are employed, the source(s) of silver and fluoride (e.g., AgF) may be dissolved in water first and then the source of iodide or thiocyanate may be added. Alternatively, each component can be dissolved in water separately and then combined to form an aqueous oral care composition (e.g., solution).

### EXAMPLES

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

TABLE 1
Materials

| Description | Source | Location |
|---|---|---|
| Silver fluoride (AgF) | Oakwood Chemical | West Columbia, SC, USA |
| Ammonium iodide (NH₄I) | Honeywell Specialty | Seelze, Germany |
| | Chemical | |
| Ammonium thiocyanate (NH₄SCN) | Alfa Aesar | Ward Hill, MA, USA |
| Guanidinium thiocyanate | Alfa Aesar | Ward Hill, MA, USA |
| Disodium hydrogen citrate | Alfa Aesar | Ward Hill, MA, USA |
| sesquihydrate | | |
| Hydrogen Peroxide (H₂O₂) | Sigma Aldrich | St Louis, MO, USA |
| Solution, 3% water solution | | |
| (USP) | | |
| Carbamide peroxide gel (22%), | 3M Company | St. Paul, MN, USA |
| (3M ™ ESPE ™ OMNI ™ | | |
| WHITE&BRITE ™ 22% | | |
| CARBAMIDE PEROXIDE TOOTH | | |
| WHITENING SYSTEM) | | |
| Sodium carbonate | EMD | Gibbstown, NJ, USA |
| Potassium phosphate monobasic | Sigma Aldrich | St Louis, MO, USA |
| Ammonium chloride (NH₄Cl) | VWR | West Chester, PA, USA |
| Potassium sulfate | J.T. Baker | Phillipsburg, NJ, USA |
| Silver iodide | Sigma Aldrich | St Louis, MO, USA |
| Silver diamine fluoride (SDF) | Elevate Oral Care | West Palm Beach, FL, USA |
| solution 38%, commercially | | |
| available as ADVANTAGE | | |
| ARREST silver diamine fluoride | | |

### Preparation of Test Surface for Stain Removal

(Teeth Stained by Treatment with Silver Containing Solution)

Extracted human teeth were disinfected and stored in water before silver compounds treatments. These teeth were carious human teeth with different size of caries. The teeth were first rinsed with water and then rinsed with artificial saliva, rinsed again with water and dried with air to remove excess water on the surface. These teeth were then treated with one of two silver containing solutions: (1) a silver iodide fluoride complex solution, (Formulation F1, described below) or (2) commercially available silver diamine fluoride solution (SDF), described in Table 1, above. To simulate a period of time experienced by a dental patient after having their teeth treated with a silver containing solution, the following procedure was followed. The treated teeth were first stored in 37° C. artificial saliva for 3 days, then the teeth were stored at 37° C. in lactic acid-based demineralization solution for one day, and finally, the teeth were stored at 37° C. in artificial saliva for 3 days. This sequence was repeated 3 times. After this sequence of treatments all the carious human teeth showed some degree of black staining, although the degree of staining differed somewhat between the different silver containing solutions and among the individual teeth. In all instances the amount of staining was greater for the teeth treated with SDF. These stained human teeth were then used in the post treatment process described below, which is the claimed subject of this disclosure, to demonstrate the removal of the black staining.

### Preparatory Solutions:

Formulation F1 (silver iodide fluoride complex solution): an aqueous oral care composition of silver iodide fluoride complex was prepared by combining 31.0 grams of silver iodide (AgI), 31.4 grams of ammonium iodide (NH₄I), 4.9 grams of ammonium fluoride (NH₄F), and 32.7 grams of water for a total weight of 100 grams of Formulation F1 solution.

Concentrated NH₄I water solution: a concentrated ammonium iodide solution was prepared by dissolving 172 gram of ammonium Iodide in 100 grams of water at room temperature, which is a saturated solution at room temperature.

Concentrated KI solution: a concentrated potassium iodide solution was prepared by dissolving 140 grams of KI in 100 grams of water at room temperature, which is a saturated solution at room temperature.

Mixture #1: 4 grams of the concentrated NH₄I water solution was mixed with 1 gram of silver iodide fluoride complex solution (Formulation F1) to form a solution.

Mixture #2: 4 gram of concentrated KI water solution was mixed with 1 gram of silver iodide fluoride complex solution (Formulation F1) to form a solution.

Diluted Hydrogen Peroxide Solution: 2 grams of 3% H₂O₂ solution was mixed with 4 grams of water to prepare a 1% (diluted) H₂O₂ solution.

### Example 1A: Treatment with 3% H₂O₂ and then Concentrated KI Solution

The prepared test surfaces, human teeth stained black due to silver treatment and conditioning described above, were first treated with 3% H₂O₂ USP water solution by rubbing the H₂O₂ on the black stain for 30 seconds, then rinsed with water and wiped off excess water on the teeth surface with paper. Next the teeth were treated with a concentrated potassium iodide for 1 minute and rinsed with water. In both cases of test surfaces: (1) teeth previously stained by treatment with a silver iodide fluoride complex solution, (Formulation F1), and (2) teeth stained with commercially available silver diamine fluoride solution (SDF), the black stains were removed after post treatment with 3% H₂O₂ as the oxidant agent and then treatment with concentrated potassium iodide (KI) solution treatments, followed by rinse with water.

### Example 1B: Treatment with 3% H₂O₂ and then Formulation F1

The prepared test surfaces, human teeth stained black due to silver treatment and conditioning described above, were first treated with 3% H₂O₂ USP water solution by rubbing the H₂O₂ on the black stain for 30 seconds, then rinsed with water and wiped off excess water on the teeth surface with paper. Next the teeth were treated with a silver iodide fluoride complex solution, (Formulation F1) for 1 minute and rinsed with water. In both cases of test surfaces: (1) teeth previously stained by treatment with a silver iodide fluoride complex solution, (Formulation F1), and (2) teeth stained with commercially available silver diamine fluoride solution (SDF), the black stains were removed after post treatment with 3% H₂O₂ as the oxidant agent then treatment with silver iodide fluoride complex solution, (Formulation F1), followed by rinse with water.

### Example 2A: Treatment with Carbamide Peroxide Gel (22%) and then Formulation F1

The prepared test surfaces, human teeth stained black due to silver treatment and conditioning described above, were first treated with carbamide peroxide gel (22%) by rubbing the H₂O₂ gel on the black stain for 30 seconds, then rinsed with water and wiped off excess water on the teeth surface with paper. Next the teeth were treated with a silver iodide fluoride complex solution, (Formulation F1) for 1 minute and rinsed with water. In both cases of test surfaces: (1) teeth previously stained by treatment with a silver iodide fluoride complex solution, (Formulation F1), and (2) teeth stained with commercially available silver diamine fluoride solution (SDF), the black stains were removed after post treatment with carbamide peroxide gel (22%) as the oxidant agent and then treatment with silver iodide fluoride complex solution, (Formulation F1), followed by rinse with water.

### Example 2B: Treatment with Carbamide Peroxide Gel (22%) and then Concentrated NH₄I Solution

The prepared test surfaces, human teeth stained black due to silver treatment and conditioning described above, were first treated with carbamide peroxide gel (22%) by rubbing the H₂O₂ gel on the black stain for 30 seconds, then rinsed with water and wiped off excess water on the teeth surface with paper. Next the teeth were treated with a concentrated ammonium iodide (NH₄I) for 1 minute and rinsed with water. In both cases of test surfaces: (1) teeth previously stained by treatment with a silver iodide fluoride complex solution, (Formulation F1), and (2) teeth stained with commercially available silver diamine fluoride solution (SDF), the black stains were removed after post treatment with carbamide peroxide gel (22%) as the oxidant agent and then treatment with concentrated ammonium iodide (NH₄I) solution, followed by rinse with water.

### Example 3A: Treatment with 3% H₂O₂ and then Mixture #1

The prepared test surfaces, human teeth stained black due to silver treatment and conditioning described above, were first treated with 3% H₂O₂ USP water solution by rubbing the H₂O₂ on the black stain for 30 seconds, then rinsed with water and wiped off excess water on the teeth surface with paper. Next the teeth were treated with Mixture #1 (an ammonium iodide/silver iodide fluoride complex solution) for 1 minute and rinsed with water. In both cases of test surfaces: (1) teeth previously stained by treatment with a silver iodide fluoride complex solution, (Formulation F1), and (2) teeth stained with commercially available silver diamine fluoride solution (SDF), the black stains were removed after post treatment with 3% H₂O₂ as the oxidant agent then treatment with Mixture #1 (an ammonium iodide/silver iodide fluoride complex solution), followed by rinse with water.

### Example 3B: Treatment with 3% H₂O₂ and then Mixture #2

The prepared test surfaces, human teeth stained black due to silver treatment and conditioning described above, were first treated with 3% H₂O₂ USP water solution by rubbing the H₂O₂ on the black stain for 30 seconds, then rinsed with water and wiped off excess water on the teeth surface with paper. Next the teeth were treated with Mixture #2 (a potassium iodide/silver iodide fluoride complex solution) for 1 minute and rinsed with water. In both cases of test surfaces: (1) teeth previously stained by treatment with a silver iodide fluoride complex solution, (Formulation F1), and (2) teeth stained with commercially available silver diamine fluoride solution (SDF), the black stains were removed after post treatment with 3% H₂O₂ as the oxidant agent then treatment with Mixture #2 (a potassium iodide/silver iodide fluoride complex solution), followed by rinse with water.

### Example 4: Treatment with 1% H₂O₂ and then Formulation F1

The prepared test surfaces, human teeth stained black due to silver treatment and conditioning described above, were first treated with 1% H₂O₂ USP water solution by rubbing the H₂O₂ on the black stain for 30 seconds, then rinsed with water and wiped off excess water on the teeth surface with paper. Next the teeth were treated with a silver iodide fluoride complex solution, (Formulation F1) for 1 minute and rinsed with water. In both cases of test surfaces: (1) teeth previously stained by treatment with a silver iodide fluoride complex solution, (Formulation F1), and (2) teeth stained with commercially available silver diamine fluoride solution (SDF), the black stains were removed after post treatment with 1% H₂O₂ as the oxidant agent then treatment with silver iodide fluoride complex solution, (Formulation F1), followed by rinse with water.

### Alternatives to Formulation F1.

The composition of Formulation F1 and suitable alternatives are presented in the tables below. The alternative formulations, F2-F15, which contain sources of iodide and/or thiocyanate, would perform in the same manner as Formulation F1 in the process of removal of stains caused by previous treatment with silver solutions, as shown in Examples above.

TABLE 2
Formulations F1 and F2

| Component | F1 | F2 |
|---|---|---|
| AgI | 31.0 | 0 |
| AgF | 0 | 18.5 |
| NH₄I | 31.4 | 0 |
| NH₄F | 4.9 | 0 |
| Guanidinium thiocyanate | 0 | 51.9 |
| DI water | 32.7 | 29.6 |
| Total % | 100 | 100 |
| Ag % | 14.3 | 15.7 |
| Total Iodine % | 44.4 | 0 |
| Total Fluoride % | 2.5 | 2.8 |

TABLE 3
Formulations F3-F7

| Component | F3 | F4 | F5 | F6 | F7 |
|---|---|---|---|---|---|
| AgF | 22.7 | 20.8 | 19.2 | 17.9 | 16.7 |
| NH₄SCN | 45.5 | 41.7 | 38.5 | 35.7 | 33.3 |
| H₂O | 31.8 | 37.5 | 42.3 | 46.4 | 50.0 |
| Total (%) | 100 | 100 | 100 | 100 | 100 |
| Ag % | 19.3 | 17.7 | 16.4 | 15.2 | 14.2 |
| Fluoride % | 3.4 | 3.1 | 2.9 | 2.7 | 2.5 |
| Ag/SCN mole ratio | 0.30 | 0.30 | 0.30 | 0.30 | 0.30 |

TABLE 4
Formulations F8-F11

| Component | F8 | F9 | F10 | F11 | F12 |
|---|---|---|---|---|---|
| AgF | 19.2 | 17.9 | 16.7 | 15.6 | 17.2 |
| NH₄I | 57.7 | 53.6 | 50.0 | 46.9 | 51.7 |
| H₂O | 23.1 | 28.6 | 33.3 | 37.5 | 31.0 |
| Total (%) | 100 | 100 | 100 | 100 | 100 |
| Ag % | 16.4 | 15.2 | 14.2 | 13.3 | 14.7 |
| Fluoride % | 2.9 | 2.7 | 2.5 | 2.3 | 2.6 |
| Ag/I mole ratio | 0.38 | 0.38 | 0.38 | 0.38 | 0.38 |

TABLE 5
Formulations F13-F15

| Component | F13 | F14 | F15 |
|---|---|---|---|
| AgF | 20.0 | 21.7 | 22.7 |
| NH₄SCN | 40.0 | 39.1 | 45.5 |
| H₂O | 40.0 | 39.1 | 31.8 |
| Total (%) | 100 | 100 | 100 |
| Ag/SCN mole ratio | 0.30 | 0.33 | 0.30 |
| Ag % | 17.0 | 18.5 | 19.3 |
| Fluoride % | 3.0 | 3.3 | 3.4 |

The complete disclosures of the patents, patent documents, and publications cited herein are incorporated by reference in their entirety as if each were individually incorporated. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. A method for at least partly removing a stain on a tooth surface, the stain comprising silver sulfide or otherwise being the result of treatment with silver diamine fluoride, the method comprising:
introducing an oxidizing composition into an oral cavity of the patient such the oxidizing composition contacts the stain on the tooth surface; and
introducing an oral care composition into the oral cavity such that the oral care composition contacts the stain on the tooth surface,
the oral care composition comprising:
a source of iodide anions, or
a source of thiocyanate anions.

2. The method of claim 1, wherein the oxidizing composition comprises peroxide.

3. The method of claim 2, wherein peroxide is present in the oxidizing composition as hydrogen peroxide, a persulfate, benzoyl peroxide, or carbamide peroxide.

4. The method of claim 2, wherein the peroxide is present in the oxidizing composition in an amount of at least 0.1 wt. %, based on the total weight of the oxidizing composition.

5. The method of claim 1, wherein the source of iodide anions is present in the oral composition as potassium iodide, ammonium iodide, sodium iodide, calcium iodide, silver iodide, or a combination thereof.

6. The method of claim 1, wherein the oral care composition comprises iodide anions, and further comprises silver cations, fluoride anions, and water.

7. The method of claim 6, wherein the oral care composition comprises 13-17 wt-% silver cations and 2.25-3.0 wt-% fluoride anions, based on the total weight of the oral care composition.

8. The method of claim 6, wherein the silver cations and the iodide anions are present in the oral care composition in a silver:iodide molar ratio of less than 0.42:1, and water is present in the oral care composition at an amount of less than 41.5 wt-%, based on the total weight of the oral care composition.

9. The method of claim 1, wherein the thiocyanate anions are present in the oral composition as ammonium thiocyanate, sodium thiocyanate, potassium thiocyanate, guanidinium thiocyanate, or a combination thereof.

10. The method of claim 1, wherein the oral care composition comprises thiocyanate anions, and further comprises silver cations, fluoride anions, and water.

11. The method of claim 10, wherein the oral care composition comprises 12.2-20 wt-% silver cations; and 2.0-4.0 wt-% fluoride anions, based on the total weight of the oral care composition.

12. The method of claim 10, wherein the silver cations and the thiocyanate anions are present in the oral care composition in a silver:thiocyanate molar ratio of at least 0.1:1 and less than 0.37:1.

13. The method of claim 1, wherein the step of introducing the oral care composition comprises contacting the tooth with the oral care composition for at least 5 seconds.

14. The method of claim 1, wherein the step of introducing the oral care composition occurs after the step of introducing the oxidizing composition.

15. The method of claim 1, wherein the step of introducing the oral care composition occurs within 60 minutes of the step of introducing the oxidizing composition.

16. (canceled)

17. The method of claim 1, the oral care composition further comprising a source of silver cations, wherein the source of silver cations are present in the oral care composition as silver iodide, silver fluoride, or a combination thereof.

18. The method of claim 17, the oral care composition further comprising one or more ammonium salt.

19. The method of claim 18, the one or more ammonium salt selected from ammonium iodide, ammonium fluoride, ammonium thiocyanate, and a combination thereof.

20. The method of claim 1,
the source of iodide anions is present in the oral care composition as silver iodide, ammonium iodide, or a combination thereof, or
the source of thiocyanate anions is present in the oral care composition as guanidium thiocyanate, ammonium thiocyanate, or a combination thereof.

21. A kit for at least partly removing a stain on a tooth surface, the stain comprising silver sulfide or otherwise being the result of treatment with silver diamine fluoride, the kit comprising:
an oxidizing composition; and
an oral care composition comprising:
a source of iodide anions, or
a source of thiocyanate anions.
